(19) United States
(12) Patent Application Publication
(10) Pub. No.: US 2024/0057862 A1
(43) Pub. Date: Feb. 22, 2024

(54) OPHTHALMOLOGIC INFORMATION PROCESSING SYSTEM, OPHTHALMOLOGIC INFORMATION PROCESSING SERVER, OPHTHALMOLOGIC INFORMATION PROCESSING PROGRAM, AND OPHTHALMOLOGIC INFORMATION PROCESSING METHOD

(71) Applicant: TOPCON CORPORATION, Itabashi-ku, Tokyo (JP)

(72) Inventor: Yoshitsugu TAKAHASHI, Tokyo (JP)

(73) Assignee: TOPCON CORPORATION, Itabashi-ku, Tokyo (JP)

(21) Appl. No.: 18/259,587

(22) PCT Filed: Dec. 22, 2021
(86) PCT No.: PCT/JP2021/047704

§ 371 Date: Jun. 28, 2023

(30) Foreign Application Priority Data
Dec. 29, 2020 (JP) 2020-219844

(51) Int. Cl. A61B 3/117; A61B 3/103; A61B 3/00; G16H 50/20; G16H 50/30

(52) U.S. Cl. CPC A61B 3/1173; A61B 3/103; A61B 3/0025; G16H 50/20; G16H 50/30; A61B 2560/02

(57) ABSTRACT

An ophthalmologic information processing system for selecting a subject at risk of glaucoma as a candidate includes: an information acquisition unit that acquires measurement information of an eye of the subject measured by an ophthalmologic apparatus; and a determination unit that determines whether the subject is a candidate at risk of glaucoma based on an anterior chamber depth of the subject included in the measurement information acquired by the information acquisition unit.

### TECHNICAL FIELD

The present disclosure relates to an ophthalmologic information processing system, an ophthalmologic information processing server, an ophthalmologic information processing program, and an ophthalmologic information processing method for selecting a subject at risk of glaucoma as a candidate.

### BACKGROUND ART

Noncontact tonometers have been used for early detection of glaucoma.

For example, Patent Document 1 discloses a known example of a noncontact tonometer. In this example, an air flow is blown to the cornea of a subject's eye to deform the cornea, and the deformation of the cornea is optically detected to measure the intraocular pressure.

### CITATION LIST

### Patent Document

Patent Document 1: Japanese Unexamined Patent Publication No. 2006-262990

### SUMMARY OF THE INVENTION

### Technical Problems

Although making no contact with the subject, the existing noncontact tonometer burdens the subject when blowing the air flow to the cornea. Early detection of glaucoma without using such tonometry has been expected.

In view of the foregoing, an object of the present invention is to provide an ophthalmologic information processing system, an ophthalmologic information processing server, an ophthalmologic information processing program, and an ophthalmologic information processing method that are capable of supporting early detection of glaucoma and reducing burdens on a subject without using tonometry.

### Solution to the Problems

To achieve the object, the present disclosure provides an ophthalmologic information processing system for selecting a subject at risk of glaucoma as a candidate. The ophthalmologic information processing system includes: an information acquisition unit that acquires measurement information of an eye of the subject measured by an ophthalmologic apparatus; and a determination unit that determines whether the subject is a candidate at risk of glaucoma based on an anterior chamber depth of the subject included in the measurement information acquired by the information acquisition unit.

According to another aspect, the present disclosure provides an ophthalmologic information processing server for selecting a subject at risk of glaucoma as a candidate. The ophthalmologic information processing server includes: an information acquisition unit that acquires measurement information of an eye of the subject measured by an ophthalmologic apparatus; and a determination unit that determines whether the subject is a candidate at risk of glaucoma based on an anterior chamber depth of the subject included in the measurement information acquired by the information acquisition unit.

According to another aspect, the present disclosure provides an ophthalmologic information processing program for selecting a subject at risk of glaucoma as a candidate. The ophthalmologic information processing program causes a computer to perform: an information acquisition step in which an information acquisition unit acquires measurement information of an eye of the subject measured by the ophthalmologic apparatus; and a determination step in which a determination unit determines whether the subject is a candidate at risk of glaucoma based on an anterior chamber depth of the subject included in the measurement information acquired by the information acquisition unit.

According to another aspect, the present disclosure provides an ophthalmologic information processing method for selecting a subject at risk of glaucoma as a candidate using an ophthalmologic apparatus and a computer. The ophthalmologic information processing method includes: an information acquisition step in which an information acquisition unit acquires measurement information of an eye of the subject measured by the ophthalmologic apparatus; and a determination step in which a determination unit determines whether the subject is a candidate at risk of glaucoma based on an anterior chamber depth of the subject included in the measurement information acquired by the information acquisition unit.

### Advantages of the Invention

The present disclosure can support early detection of glaucoma and can reduce burdens on a subject without using tonometry.

### BRIEF DESCRIPTION OF THE DRAWINGS

FIG. 1 is a system configuration diagram of an ophthalmologic information processing system of an embodiment of the present disclosure.

FIG. 2 is a configuration diagram of an ophthalmologic apparatus.

FIG. 3 is a schematic view illustrating an anterior chamber depth and an eye axial length.

FIG. 4 is a diagram showing examples of determination of a subject at risk of glaucoma by a determination unit.

FIG. 5 is a flowchart illustrating a main flow of a process.

### DETAILED DESCRIPTION

(System Configuration)

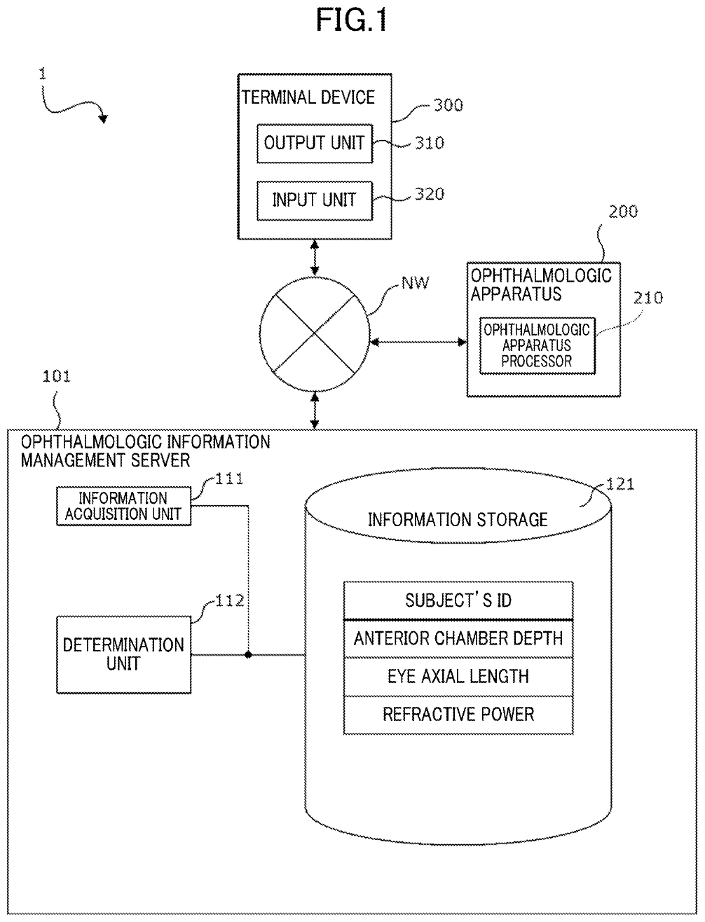

An embodiment of the present disclosure will be described below with reference to the drawings. FIG. 1 is a system configuration diagram of an ophthalmologic information processing system 1 of an embodiment of the present disclosure. This drawing shows a configuration example in which the ophthalmologic information processing system 1 includes an information management server 101, an ophthalmologic apparatus 200, and a terminal device 300 that are different apparatuses and are connected to each other via a network NW. Note that the ophthalmologic information processing system 1 may be a stand alone system including all the components integrated together. Alternatively, the function of the ophthalmologic information processing system 1 of selecting a subject at risk of glaucoma may be provided as one of modes of the ophthalmologic apparatus 200. The ophthalmologic apparatus 200 functions as a measuring apparatus that measures and acquires the anterior chamber depth, the eye axial length, and the refractive power so that the ophthalmologic information processing system 1 can select a subject at risk of glaucoma as a candidate. The ophthalmologic information processing system 1 may be configured not to directly require the ophthalmologic apparatus 200 in the step of selection by acquiring, and storing therein, the anterior chamber depth, the eye axial length, and the refractive power from another external ophthalmologic apparatus 200.

In the ophthalmologic information processing system 1 shown in FIG. 1, the information management server 101 and the terminal device 300 to be used by a user are communicably connected to each other via the network NW. The network NW is, for example, the Internet or a virtual private network (VPN). Although FIG. 1 shows the terminal device 300 alone that is supposed to be handled by a single user for the sake of simple illustration, the information management server 101 can be connected to two or more terminal devices, i.e., two or more users, via the network NW. A single terminal device may be handled by two or more users.

The ophthalmologic information processing system 1 measures the subject's eye with the ophthalmologic apparatus 200 to automatically select a subject at risk of glaucoma as a candidate for diagnosis of glaucoma mainly by an ophthalmologist based on the acquired information. The diagnosis of glaucoma is conducted by the ophthalmologist, and the ophthalmologic information processing system 1 of the embodiment of the present disclosure is used for preliminary and auxiliary selection, i.e., so-called screening. An operating company of the ophthalmologic information processing system 1 provides services for supporting, for example, the diagnosis of the ophthalmologist using the system. The operating company can provide the services for multiple ophthalmologists or others. The operating company of the ophthalmologic information processing system 1 manages the information management server 101, and the ophthalmologist or others uses the terminal device 300 and the ophthalmologic apparatus 200. The ophthalmologists themselves may manage the information management server 101. An apparatus for acquiring measurement information on the anterior chamber depth, the eye axial length, and the refractive power, which will be described later, may be a measuring apparatus having the measurement function similar to that of the ophthalmologic apparatus 200. The measurement is conducted not only by or at ophthalmologic hospitals, but by a measuring apparatus installed in a place such as a glasses store.

The terminal device 300 is a device such as a personal computer (PC), a smartphone, a tablet PC, or a mobile phone. The terminal device 300 may access the information management server 101 via dedicated application software installed in the terminal device. Alternatively, the terminal device 300 may access the information management server 101 using an operating environment (e.g., an application programming interface (API), a cloud service, or a platform) provided by the information management server 101.

An input unit 320 is, for example, a device that is handled by a user to input and select information, such as a keyboard, a mouse, a trackball, or a touchpad. The input unit 320 may be a touch screen integrated with an output unit 310 such as a liquid crystal display or organic EL display of a smartphone, a tablet, or a PC. The input unit 320 may be a voice input device.

The output unit 310 is, for example, a display having a screen unit for displaying information or the like to the user. A display independent of the terminal device 300 may be used, or a display such as a liquid crystal display or an organic EL display of a smartphone or a tablet may be used.

The ophthalmologic apparatus 200 is mainly used to acquire measurement information about the anterior chamber depth, the eye axial length, and the refractive power. The ophthalmologic apparatus 200 of this example can measure all of the anterior chamber depth, the eye axial length, and the refractive power by itself. Since the ophthalmologic information processing system 1 includes, as one of the components, the ophthalmologic apparatus 200 capable of measuring all the measurement information, it is suitably possible to acquire the measurement information of the subject's eye and select a subject at risk of glaucoma immediately.

The ophthalmologic apparatus 200 may be capable of measuring not only the anterior chamber depth, eye axial length, and refractive power of the eye of the subject, but also other items such as a pupillary distance, a corneal curvature, and a corneal diameter. The anterior chamber depth and the eye axial length can be measured by the function of, for example, an optical coherence tomography (OCT) or a noncontact optical eye axial length measuring apparatus included in the ophthalmologic apparatus 200. The refractive power can be measured by the function of, for example, an automatic refractometer included in the ophthalmologic apparatus 200. It is suitable to use the ophthalmologic apparatus 200 which achieves these measurement functions by one measuring apparatus.

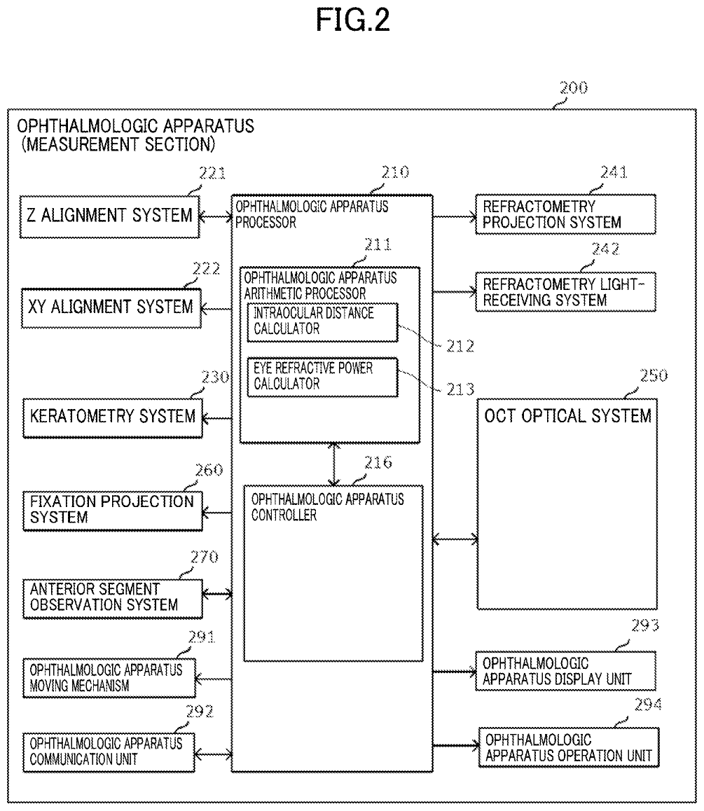

FIG. 2 shows a schematic configuration of the ophthalmologic apparatus 200. The ophthalmologic apparatus 200 includes an ophthalmologic apparatus processor 210, an ophthalmologic apparatus arithmetic processor 211, an intraocular distance calculator 212, an eye refractive power calculator 213, an ophthalmologic apparatus controller 216, a Z alignment system 221, an XY alignment system 222, a keratometry system 230, a refractometry projection system 241, a refractometry light-receiving system 242, an OCT optical system 250, a fixation photographing system 260, an anterior segment observation system 270, an ophthalmologic apparatus moving mechanism 291, an ophthalmologic apparatus communication unit 292, an ophthalmologic apparatus display unit 293, and an ophthalmologic apparatus operation unit 294. Note that components and details that are less relevant to the functions of the ophthalmologic information processing system 1 of the present disclosure will not be described below.

The intraocular distance calculator 212 analyzes the result of detection of interfering light by the OCT optical system 250 to specify peak positions of the detection result of the interfering light corresponding to a predetermined site in the eye, and thus can obtain the anterior chamber depth and eye axial length of the subject's eye based on the distance between the specified peak positions.

The eye refractive power calculator 213 analyzes a ring-shaped image (a pattern image) obtained when an imaging element of the refractometry light-receiving system 242 receives reflection of a ring-shaped luminous flux (a ring-shaped measurement pattern) projected on the fundus of the eye by the refractometry projection system 241. Thus, the refractive power can be obtained.

The information management server 101 includes an information acquisition unit 111, a determination unit 112, and an information storage 121.

The information storage 121 stores an ID of the subject and items of information associated with the ID, such the anterior chamber depth, the eye axial length, and the refractive power.

The ID of the subject may be information indicating, for example, the name of the subject, the number of a health insurance card, or the number of a patient ID card unique to a hospital. The anterior chamber depth, the eye axial length, and the refractive power include data for each of the left and right eyes of the subject. Information about measurement timing of each data may include information about measurement date including year, month, and day when the eye axial length of the subject was measured and time when the measurement was made on the measurement date.

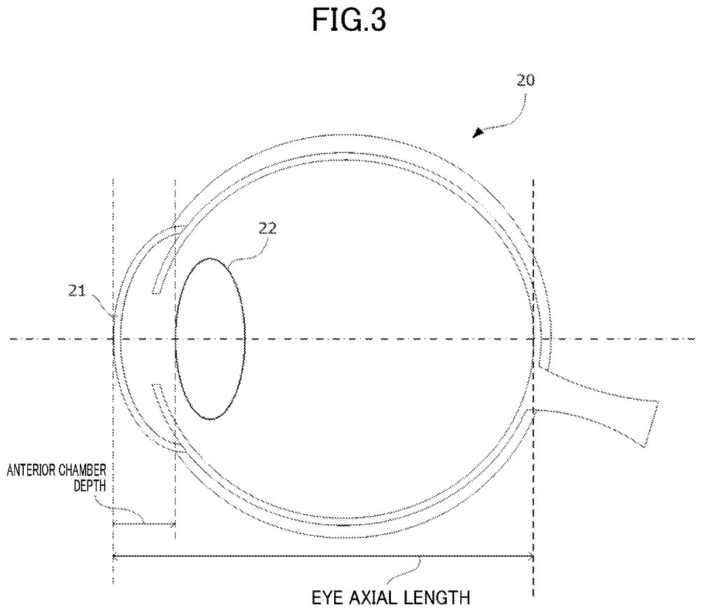

FIG. 3 is a schematic view illustrating the anterior chamber depth and the eye axial length. As shown in this drawing, the anterior chamber depth is a distance from the cornea 21 to the crystalline lens 22 and is expressed in millimeters (mm), for example. The eye axial length is a distance from the cornea 21 to the retina and is expressed in millimeters (mm) or centimeters (cm). The refractive power is expressed in diopters (D). The larger value typically indicates the greater refractive power. For example, a refractive power of −4D is greater than a refractive power of −2D.

(Function of Selecting Subject at Risk of Glaucoma)

The components of the present disclosure will be described below to describe the function of selecting a subject at risk of glaucoma.

The information acquisition unit 111 has the function of acquiring measurement information of the eye of the subject measured by the ophthalmologic apparatus 200. For example, the information acquisition unit 111 acquires information on the anterior chamber depth and the eye axial length calculated by the intraocular distance calculator 212 of the ophthalmologic apparatus arithmetic processor 211 and the refractive power calculated by the eye refractive power calculator 213 as a result of the measurement, via the ophthalmologic apparatus communication unit 292, the network NW, or the like.

The determination unit 112 has the function of determining whether the subject is a candidate at risk of glaucoma based on the anterior chamber depth, the eye axial length, and the refractive power included in the measurement information acquired by the information acquisition unit 111.

Specifically, numerical values of the anterior chamber depth, eye axial length, and refractive power of the subject's eye measured by the ophthalmologic apparatus 200 are compared with predetermined reference numerical values to determine whether each of these values is satisfactory or not, and whether the subject is a candidate at risk of glaucoma is determined based on the combinations of the results of the determination of these items. Based on the determination results, an ophthalmologist or any other suitable person diagnoses whether the subject is at risk of glaucoma, and thus can take the subsequent actions, such as a follow-up care, other examinations, or treatments.

Examples of the items and criteria for determining whether the values of the items are satisfactory or not will be described below. The determination unit 112 places the greatest importance on whether the anterior chamber depth, among the items related to the subject, is satisfactory or not. In a preferred example, a criterion for the anterior chamber depth is whether the anterior chamber depth is, for example, less than 2.0 mm. Although the reference numerical value for each item can be set as appropriate, the quantitative criterion that is suitably set as described above allows clearer determination. This is because when the anterior chamber depth is less (shallower) than a certain value such as 2.0 mm, the intraocular pressure increases, raising the possibility of developing glaucoma. The determination unit 112 compares the anterior chamber depth of the subject with the reference value of the anterior chamber depth, and outputs information indicating that the anterior chamber depth of the subject is determined not to satisfy the criterion when the anterior chamber depth is small (shallow). The subject can be determined to be at risk of glaucoma even when only the anterior chamber depth does not satisfy the criterion.

Reference is also made to whether the eye axial length, which is another item, is satisfactory or not. In a preferred example, a criterion for the eye axial length is whether the eye axial length is, for example, equal to or more than 26.5 mm. This is because when the eye axial length is equal to or more (longer) than a certain value such as 26.5 mm, myopia is progressing, and myopia is considered to be one of risk factors for glaucoma. The determination unit 112 compares the eye axial length of the subject with the reference value of the eye axial length, and outputs information indicating that the eye axial length of the subject is determined not to satisfy the criterion when the eye axial length is great (long). In a preferred example, the eye axial length is used as an auxiliary item that improves the reliability of the determination using the anterior chamber depth as the most important factor. Thus, the subject should not be determined to be at risk of glaucoma when only the eye axial length does not satisfy the criterion. In a preferred example, the determination is made by referring to the anterior chamber depth and the eye axial length in combination. For example, the subject can be comprehensively determined to be at risk of glaucoma when both of the anterior chamber depth and the eye axial length do not satisfy the criteria. It is thus possible to select the subject at risk of glaucoma more accurately by referring to the other items than the anterior chamber depth.

Reference is also made to whether the refractive power, which is another item, is satisfactory or not. In a preferred example, a criterion for the refractive power is whether the refractive power is higher than, for example, −8D. This is because when the refractive power exceeds (higher than) a predetermined reference value such as −8D, myopia is progressing, and myopia is considered to be one of risk factors for glaucoma. The determination unit 112 compares the refractive power of the subject with the reference value of the refractive power, and outputs information indicating that the refractive power of the subject is determined not to satisfy the criterion when the refractive power of the subject is high. In a preferred example, just like the eye axial length, the refractive power is also used as an auxiliary item that improves the reliability of the determination using the anterior chamber depth as the most important factor. Thus, the subject should not be determined to be at risk of glaucoma when only the refractive power does not satisfy the criterion. In a preferred example, the determination is made by referring to the anterior chamber depth and the refractive power in combination, or the anterior chamber depth, the eye axial length, and the refractive power in combination. For example, the subject can be comprehensively determined to be at risk of glaucoma when both of the anterior chamber depth and the refractive power do not satisfy the criteria. Alternatively, the subject can be comprehensively determined to be at risk of glaucoma when the anterior chamber depth, the eye axial length, and the refractive power do not satisfy the criteria.

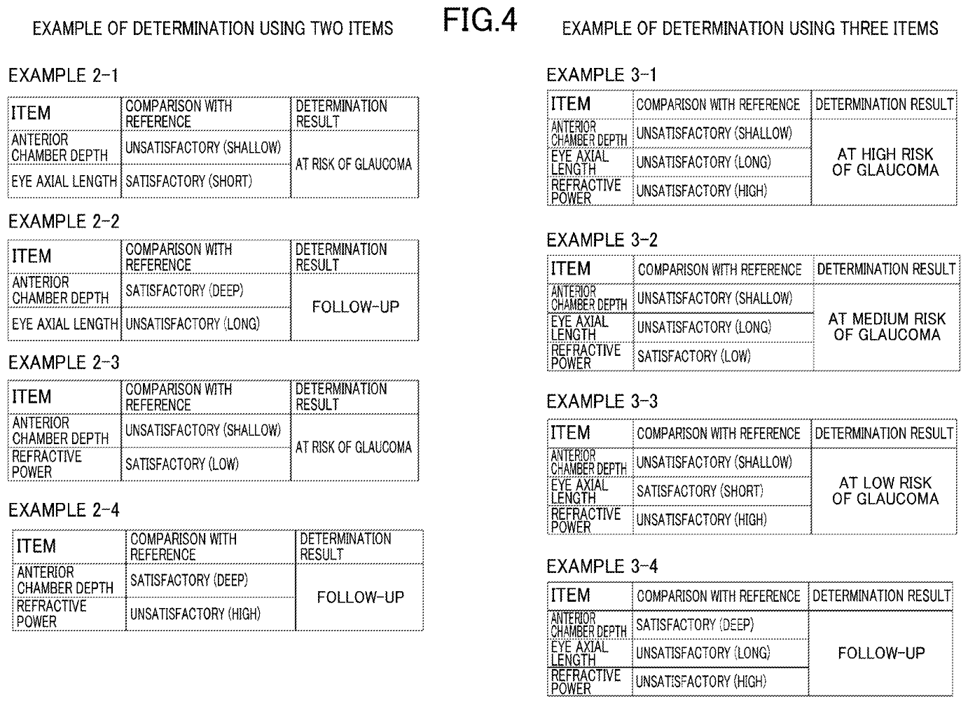

FIG. 4 is a diagram illustrating examples of logic for the determination by the determination unit 112 as to whether a subject is at risk of glaucoma using the anterior chamber depth, the eye axial length, and the refractive power. The ophthalmologic information processing system, ophthalmologic information processing server, ophthalmologic information processing program, and ophthalmologic information processing method of the embodiment of the present disclosure can determine whether the subject is at risk of glaucoma by using the items measured by the ophthalmologic apparatus 200 as they are. In particular, it is possible to determine whether the subject is at risk of glaucoma based on multiple items by using the ophthalmologic apparatus 200 that can measure all of the anterior chamber depth, the eye axial length, and the refractive power. An example of the determination using the multiple items will be described below.

As shown in FIG. 4, in a case of using two items including the anterior chamber depth and one of the eye axial length or the refractive power for the determination, the subject can be determined to be at risk of glaucoma when the anterior chamber depth and one of the eye axial length or the refractive power do not satisfy the criteria, for example, as shown in Examples 2-1 and 2-3, for improved reliability of the determination. The determination can be made with higher accuracy by considering not only the anterior chamber depth but also the factors related to myopia. Further, it may be determined that the subject is not selected as a candidate at risk of glaucoma at this point and that a follow-up with the subject is selected as the subsequent action when the anterior chamber depth satisfies the criterion although one of the eye axial length or the refractive power does not satisfy the criterion, as can be seen in Examples 2-2 and 2-4. This is because when the anterior chamber depth satisfies the criterion and the item related to myopia does not satisfy the criterion, it may be simply that the subject has myopia.

In a case of using all the three items of the anterior chamber depth, the eye axial length, and the refractive power for the determination as shown in Examples 3-1, 3-2, and 3-3, the determination may be made by stepwise evaluation, for example, the subject is at high, medium, or low risk of glaucoma. For example, in Example 3-1, the anterior chamber depth does not satisfy the criterion, neither do the eye axial length and the refractive power. Thus, the determination can be made with higher accuracy by referring to both of the eye axial length related to axial myopia and the refractive power related to refractive myopia. In comparison between Examples 3-2 and 3-3, a high priority is given to the eye axial length than to the refractive power as the auxiliary item, and thus it can be determined that the risk of glaucoma is lower in Example 3-3. Further, it may be determined that the subject is not selected as a candidate at risk of glaucoma at this point and that a follow-up with the subject is selected as the subsequent action when the anterior chamber depth satisfies the criterion although both of the eye axial length and the refractive power do not satisfy the criterion, as can be seen in Example 3-4. This is because when the anterior chamber depth satisfies the criterion and the items related to myopia do not satisfy the criterion, it may be simply that the subject has myopia.

(Process Flow)

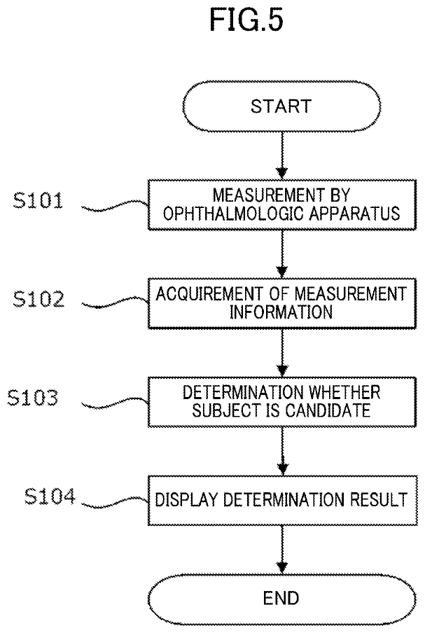

FIG. 5 is a flowchart illustrating a main flow of a process executed by the information management server 101 of the ophthalmologic information processing system 1 of the embodiment of the present disclosure. An ophthalmologic information processing method will be described below with reference to the flowchart. The flowchart is merely an example, and the ophthalmologic information processing method is not limited to the process of the flowchart.

In Step S101, the ophthalmologic apparatus 200 measures the subject's eye to obtain measurement information.

In Step S102, the information acquisition unit 111 acquires the measurement information of the subject's eye measured by the ophthalmologic apparatus 200.

In Step S103, the determination unit 112 determines whether the subject is a candidate at risk of glaucoma based on the anterior chamber depth, eye axial length, and refractive power of the subject's eye acquired by the information acquisition unit 111.

In Step S104, the result of the determination by the determination unit 112 as to whether the subject is a candidate at risk of glaucoma is outputted to the output unit 310 of the terminal device 300. An ophthalmologist or any other suitable person can consider the treatment to be given to the subject by referring to the outputted information.

As described above, according to the ophthalmologic information processing system, ophthalmologic information processing server, ophthalmologic information processing program, and ophthalmologic information processing method of the embodiment of the present disclosure, the ophthalmologic information processing system 1 that selects a subject at risk of glaucoma as a candidate includes: the information acquisition unit 111 that acquires measurement information of the eye of the subject measured by the ophthalmologic apparatus 200; and the determination unit 112 that determines whether the subject is a candidate at risk of glaucoma based on the anterior chamber depth of the subject included in the measurement information acquired by the information acquisition unit 111. This can support early detection of glaucoma and can reduce burdens on a subject without using tonometry, unlike the existing techniques.

The determination unit 112 determines that the subject is a candidate at risk of glaucoma when the anterior chamber depth of the subject is less than 2 mm. Thus, the determination can be made more clearly by setting an appropriate quantitative criterion.

The determination unit 112 determines whether the subject is a candidate at risk of glaucoma by additionally referring to the eye axial length of the subject included in the measurement information. It is thus possible to select a subject at risk of glaucoma more accurately by referring to other items than the anterior chamber depth.

The determination unit 112 determines that the subject is a candidate at risk of glaucoma when the eye axial length of the subject is equal to or more than 26.5 mm. Thus, the determination can be made more clearly by setting an appropriate quantitative criterion.

The determination unit 112 determines whether the subject is a candidate at risk of glaucoma by additionally referring to the refractive power of the eye of the subject included in the measurement information. It is thus possible to select a subject at risk of glaucoma more accurately by referring to other items than the anterior chamber depth.

When the refractive power of the subject exceeds −8D, the determination unit 112 determines that the subject is a candidate at risk of glaucoma. Thus, the determination can be made more clearly by setting an appropriate quantitative criterion.

(Program)

FIG. 6 is a schematic block diagram showing the configuration of a computer 801. The computer 801 includes a CPU 802, a main storage device 803, an auxiliary storage device 804, and an interface 805.

Details of a program that implements the functions constituting the information management server 101 of the embodiments will be described below.

The information management server 101 is implemented on the computer 801. The operation of the components of the information management server 101 is stored in the auxiliary storage device 804 in the form of a program. The CPU 802 reads out the program from the auxiliary storage device 804 and loads the program into the main storage device 803 to perform the above-described processing according to the program. Further, the CPU 802 reserves a storage region corresponding to the above-described storage in the main storage device 803 according to the program.

Specifically, the program is an ophthalmologic information processing program for selecting a subject having glaucoma as a candidate by the computer 801. The program achieves the function of causing the computer to perform: an information acquisition step in which the information acquisition unit 111 acquires measurement information of the eye of the subject measured by the ophthalmologic apparatus 200; and a determination step in which the determination unit 112 determines whether the subject is a candidate at risk of glaucoma based on the anterior chamber depth of the subject included in the measurement information acquired by the information acquisition unit 111.

It should be noted that the auxiliary storage device 804 is an example of a non-transitory tangible medium. Other examples of such a non-transitory tangible medium include a magnetic disk, a magneto-optical disk, a CD-ROM, a DVD-ROM, and a semiconductor memory which are connected through an interface 805. When the program is delivered to the computer 801 through the network NW, the computer 801 that has received the program may load the program into the main storage device 803 and execute the above-described process.

The program may achieve some of the above-described functions. Further, the program may be a so-called differential file (differential program) that achieves the above-described functions in combination with another program already stored in the auxiliary storage device 804.

The embodiment of the present disclosure has been described above, but the aspects of the present disclosure are not limited to the embodiment.

### DESCRIPTION OF REFERENCE CHARACTERS

101 Information Management Server
111 Information Acquisition Unit
112 Determination Unit
121 Information Storage
200 Ophthalmologic Apparatus
210 Ophthalmologic Apparatus Processor
211 Ophthalmologic Apparatus Arithmetic Processor
212 Intraocular Distance Calculator
213 Eye Refractive Power Calculator
216 Ophthalmologic Apparatus Controller
221 Z Alignment System
222 XY Alignment System
230 Keratometry System
241 Refractometry Projection System
242 Refractometry Light-Receiving System
250 OCT Optical System
260 Fixation Projection System
270 Anterior Segment Observation System
291 Ophthalmologic Apparatus Moving Mechanism
292 Ophthalmologic Apparatus Communication Unit
293 Ophthalmologic Apparatus Display Unit
294 Ophthalmologic Apparatus Operation Unit
300 Terminal Device
310 Output Unit
320 Input Unit
NW Network

1. An ophthalmologic information processing system for selecting a subject at risk of glaucoma as a candidate, the ophthalmologic information processing system comprising:
an information acquisition unit that acquires measurement information of an eye of the subject measured by an ophthalmologic apparatus; and
a determination unit that determines whether the subject is a candidate at risk of glaucoma based on an anterior chamber depth of the subject included in the measurement information acquired by the information acquisition unit.

2. The ophthalmologic information processing system of claim 1, wherein
the determination unit determines that the subject is the candidate at risk of glaucoma when the anterior chamber depth of the subject is less than 2 mm.

3. The ophthalmologic information processing system of claim 1, wherein
the determination unit determines whether the subject is the candidate at risk of glaucoma by additionally referring to an eye axial length of the subject included in the measurement information.

4. The ophthalmologic information processing system of claim 3, wherein
the determination unit determines that the subject is the candidate at risk of glaucoma when the eye axial length of the subject is equal to or more than 26.5 mm.

5. The ophthalmologic information processing system of claim 1, wherein
the determination unit determines whether the subject is the candidate at risk of glaucoma by additionally referring to a refractive power of the eye of the subject included in the measurement information.

6. The ophthalmologic information processing system of claim 5, wherein
the determination unit determines that the subject is the candidate at risk of glaucoma when the refractive power of the eye of the subject exceeds −8D.

7. An ophthalmologic information processing server for selecting a subject at risk of glaucoma as a candidate, the ophthalmologic information processing server comprising:
an information acquisition unit that acquires measurement information of an eye of the subject measured by an ophthalmologic apparatus; and
a determination unit that determines whether the subject is a candidate at risk of glaucoma based on an anterior chamber depth of the subject included in the measurement information acquired by the information acquisition unit.

8. An ophthalmologic information processing program for selecting a subject at risk of glaucoma as a candidate, the ophthalmologic information processing program causing a computer to perform:
an information acquisition step in which an information acquisition unit acquires measurement information of an eye of the subject measured by an ophthalmologic apparatus; and
a determination step in which a determination unit determines whether the subject is a candidate at risk of glaucoma based on an anterior chamber depth of the subject included in the measurement information acquired by the information acquisition unit.

9. An ophthalmologic information processing method for selecting a subject at risk of glaucoma as a candidate using an ophthalmologic apparatus and a computer, the ophthalmologic information processing method comprising:
an information acquisition step in which an information acquisition unit acquires measurement information of an eye of the subject measured by the ophthalmologic apparatus; and
a determination step in which a determination unit determines whether the subject is a candidate at risk of glaucoma based on an anterior chamber depth of the subject included in the measurement information acquired by the information acquisition unit.
